(19) United States
(12) Patent Application Publication
(10) Pub. No.: US 2022/0076829 A1
(43) Pub. Date: Mar. 10, 2022

(54) METHOD AND APPARATUS FOR ANALYZING MEDICAL IMAGE DATA IN A LATENT SPACE REPRESENTATION

(71) Applicant: Delineo Diagnostics, Inc., Williston, FL (US)

(72) Inventors: Scott Anderson MIDDLEBROOKS, Oud-Turnhout (BE); Adrianus Cornelis KOOPMAN, Hilversum (NL); Ari David GOLDBERG, Chicago, IL (US); Henricus Wilhelm VAN DER HEIJDEN, Turnhout (BE)

(73) Assignee: Delineo Diagnostics, Inc., Williston, FL (US)

(21) Appl. No.: 17/016,930

(22) Filed: Sep. 10, 2020

(51) Int. Cl. G16H 50/20; G16H 30/40; G06N 3/04; G06N 3/08

(52) U.S. Cl. CPC G16H 50/20; G06N 3/08; G06N 3/0454; G16H 30/40

(57) ABSTRACT

Disclosed are methods and systems for processing medical image data. The method comprising inputting, with one or more processors of one or more computation devices, medical image data into an encoder stage of an encoder-decoder pair (EDP) as a first input among one or more inputs; calculating, with the one or more processors, a latent space representation of the one or more inputs using the encoder stage of the EDP; providing, from a latent space database stored within one or more storage devices accessible by the one or more computation devices, latent space representations of other inputs; and determining, with the one or more processors, a classification based on the latent space representation of the one or more inputs and at least one latent space representation of the other inputs.

### FIELD OF THE DISCLOSURE

The disclosure relates to computer-aided diagnosis (CAD). The disclosure also relates to a method and a platform or system for using machine learning algorithms for processing medical data. In particular, the disclosure relates to a method and apparatus for encoding medical image data into a latent space representation and analysing said representation.

### BACKGROUND OF THE DISCLOSURE

Advances in computed tomography (CT) allow early detection of cancer, in particular lung cancer which is one of the most common cancers. As a result, there is increased focus on using regular low-dose CT screenings to ensure early detection of the disease with improved chances of success of the following treatment. This increased focus leads to an increased workload for professionals such as radiologists who have to analyze the CT screenings.

To cope with the increased workload, computer-aided detection (CADe) and computer-aided diagnosis (CADx) systems are being developed. Hereafter both types of systems will be referred to as CAD systems. CAD systems can detect lesions (e.g. nodules) and subsequently classify them as malignant or benign. A classification need not be binary, it can also include a stage of the cancer. Usually, a classification is accompanied with a confidence value as calculated by the CAD system.

CAD systems typically follow a number of general steps. In an optional first step, the input imaging data is segmented, for example to distinguish lung tissue from the background signal. Then, regions of interest are identified, for example all lung tissue with nodule-like forms in them. It is also possible to simply examine every data point, without a pre-selection of region of interest. For a selected data point a number of input values is calculated, the so-called feature vector. This feature vector is used as input in a decision function, which projects the feature vector to a classification.

Hereafter the term “model” will be used to indicate a computational framework for performing one or more of a segmentation and a classification of imaging data. The segmentation, identification of regions of interest, and/or the classification may involve the use of a machine learning (ML) algorithm. The model comprises at least one decision function, which may be based on a machine learning algorithm, which projects the input to an output. Where the term machine learning is used, this also includes further developments such as deep (machine) learning and hierarchical learning.

Whichever type of model is used, suitable training data needs to be available to train the model. In addition, there is a need to obtain a confidence value to be able to tell how reliable a model outcome is. Most models will always give a classification, but depending on the quality of the model and the training set, the confidence of the classification may vary. It is of importance to be able to tell whether or not a classification is reliable.

While CT was used as an example in this introduction, the disclosure can also be applied to other modalities, such as ultrasound, Magnetic Resonance Imaging (MRI), Positron Emission Spectrograph (PET), Single Photon Emission Computed Tomography (SPECT), X-Ray, and the like.

### SUMMARY OF THE DISCLOSURE

It is an object of this disclosure to provide a method and apparatus for classifying imaging data which addresses at least one of the above drawbacks.

Accordingly, the disclosed subject matter provides a computer-implemented method for processing medical image data, the method comprising:
inputting, with one or more processors of one or more computation devices, medical image data into an encoder stage of an encoder-decoder pair (EDP) as a first input among one or more inputs;
calculating, with the one or more processors, a latent space representation of the one or more inputs using the encoder stage of the EDP;
providing, from a latent space database stored within one or more storage devices accessible by the one or more computation devices, latent space representations of other inputs; and
determining, with the one or more processors, a classification based on the latent space representation of the one or more inputs and at least one latent space representation of the other inputs.

In an embodiment, the method comprises inputting, with the one or more processors, patient metadata into the encoder stage of the EDP as second input.

In an embodiment, the patient metadata and the calculated latent space representation are added to the latent space database stored within the one or more storage devices.

In an embodiment, the method further comprises projecting, with the one or more processors, the latent space representation using a projection function to obtain a classification value.

In an embodiment, the projection function uses a convolutional neural network (CNN).

In an embodiment, the method further comprises determining, with the one or more processors, if the latent space representation is part of a cluster of other latent space representations using a cluster detection algorithm.

In an embodiment, the EDP is a variational autoencoder. The EDP may be trained using a loss function based on a Generative Adversarial Network (GAN).

In an embodiment, a plurality of medical image data over time are used as input, and the corresponding latent space representations are tracked over time.

The disclosure further provides a computer-implemented method of training a model using medical image data, the method comprising:
inputting, with one or more processors of one or more computation devices, medical image data into an encoder-decoder pair (EDP) as first input among one or more inputs;
training, with the one or more processors, the EDP to reproduce the one or more inputs, whereby between an encoder part of the EDP and a decoder part of the EDP, the encoded data is represented in a latent space; and
further training, with the one or more processors, a projection function for projecting the latent space representation to a classification value,
wherein a loss function of the EDP training is based on the projected classification.

In an embodiment, patient metadata is input into the EDP as second input. The patient metadata may be included as embedding layer.

In an embodiment, the projection function uses a convolutional neural network (CNN).

The disclosure further provides a computing system for processing medical image data, comprising:

one or more computation devices in a computing environment and one or more storage devices accessible by the one or more computation devices, wherein the one or more computing devices comprise one or more processors, and wherein the one or more processors are programmed to:

input medical image data into an encoder stage of an encoder-decoder pair (EDP) as a first input among one or more inputs;

calculate a latent space representation based on the one or more inputs using the encoder stage of the EDP;

provide, from a latent space database stored within the one or more storage devices, latent space representations of other inputs; and

determine a classification based on the latent space representation of the one or more inputs and at least one latent space representation of the other inputs. In an embodiment, the computing devices are cloud computing devices.

In an embodiment, the system comprises a second input module for inputting patient metadata into the encoder stage of the EDP as second input among the one or more inputs.

In an embodiment, the system is configured to use a cluster detection algorithm to determine if the latent space representation is part of a cluster of other latent space representations.

In an embodiment, the EDP is a variational autoencoder. The EDP may be trained using a loss function based on a Generative Adversarial Network (GAN). The EDP may comprise a probabilistic U-Net.

In an embodiment, the system comprises a temporal analyzer for tracking a latent space representation of the one or more inputs over time.

The invention provides a computer program product comprising instructions which, when executed on a processor, cause said processor to implement one of the methods or systems as described above.

The invention further provides a non-transitory computer-readable medium with instructions stored thereon, that when executed by one or more processors, perform the steps comprising:

inputting medical image data into an encoder stage of an encoder-decoder pair (EDP) as a first input among one or more inputs;

calculating a latent space representation of the one or more inputs using the encoder stage of the EDP;

providing from a latent space database latent space representations of other inputs; and

determining a classification based on the latent space representation of the one or more inputs and at least one latent space representation of the other inputs.

### BRIEF DESCRIPTION OF THE FIGURES

Embodiments of the present disclosure will be described hereinafter, by way of example only, with reference to the accompanying drawings which are schematic in nature and therefore not necessarily drawn to scale. Furthermore, like reference signs in the drawings relate to like elements.

FIG. 1 schematically shows an overview of a workflow according to embodiments of the disclosed subject matter;

FIG. 2 schematically shows an encoder-decoder pair according to an embodiment of the disclosed subject matter;

FIG. 3 schematically shows a model according to an embodiment of the disclosed subject matter;

FIG. 4a-c schematically shows encoder-decoder pair components according to embodiments of the disclosed subject matter;

FIGS. 5a and 5b schematically show method for inputting patient metadata according to an embodiment of the disclosed subject matter;

FIG. 6 schematically shows a latent space explorer module according to an embodiment of the disclosed subject matter;

FIG. 7a-d schematically shows an example latent space according to embodiments of the disclosed subject matter;

FIG. 8 schematically shows a method for calculating confidence values according to an embodiment of the disclosed subject matter;

FIG. 9 discloses a decoder stage of an encoder-decoder pair according to an embodiment of the disclosed subject matter; and

FIG. 10 schematically shows a method for iteratively enhancing the model, according to an embodiment of the disclosed subject matter.

### DETAILED DESCRIPTION

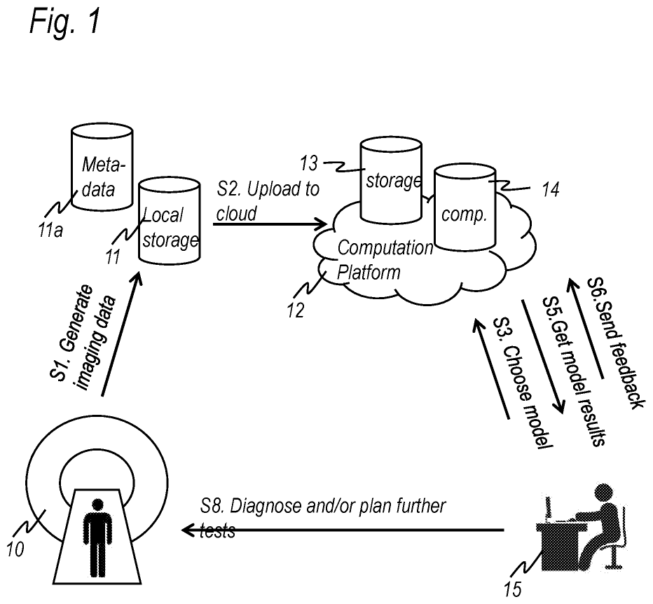

FIG. 1 schematically shows an overview of a workflow according to embodiments of the disclosed subject matter. A patient is scanned in scanning device 10. The scanning device 10 can be any type of device for generating diagnostic image data, for example an X-Ray device, a Magnetic Resonance Imaging (MRI) scanner, PET scanner, SPECT device, or any general Computed Tomography (CT) device. Of particular interest are low-dose X-Ray devices for regular and routine scans. The various types of scans can be further characterized by the use of a contrast agent, if any. The image data is typically three-dimensional (3D) data in a grid of intensity values, for example 512×512×256 intensity values in a rectangular grid.

In the following, the example of a CT device, in particular a CT device for low dose screenings, will be used. However, this is only exemplary. Aspects of the disclosure can be applied to any instantiation of imaging modality, provided that it is capable of providing imaging data. A distinct type of scan (X-Ray CT, low-dose X-Ray CT, CT with contrast agent X) can be defined as a modality.

The images generated by the CT device 10 (hereafter: imaging data) are sent to a storage 11 (step S1). The storage 11 can be a local storage, for example close to or part of the CT device 10. It can also be part of the IT infrastructure of the institute that hosts the CT device 10. The storage 11 is convenient but not essential. The data could also be sent directly from the CT device 10 to computation platform 12.

All or parts of the imaging data is then sent to the computation platform 12 in step S2. In general it is most useful to send all acquired data, so that the computer models of platform 12 can use all available information. However, partial data may be sent to save bandwidth, to remove redundant data, or because of limitations on what is allowed to be sent (e.g. because of patient privacy considerations). The data sent to the computation platform 12 may be provided with metadata from scanner 10, storage 11, or further database 11a. Metadata can include additional data related to the imaging data. For example statistical data of the patient (gender, age, medical history) or data concerning the equipment used (type and brand of equipment, scanning settings, etc).

Computation platform 12 comprises one or more storage devices 13 and one or more computation devices 14, along with the necessary network infrastructure to interconnect the devices 13, 14 and to connect them with the outside world, preferably via the Internet. It should be noted that the term “computation platform” is used to indicate a convenient implementation means (e.g. via available cloud computing resources). However, embodiments of the disclosure may use a “private platform”, i.e. storage and computing devices on a restricted network, for example the local network of an institution or hospital. The term “computation platform” as used in this application does not preclude embodiments of such private implementations, nor does it exclude embodiments of centralized or distributed (cloud) computing platforms.

The imaging data is stored in the storage 13. The central computing devices 14 can process the imaging data to generate feature data as input for the models. The computing devices 14 can segment imaging data. The computing devices 14 can also use the models to classify the (segmented) imaging data. More functionality of the computing devices 14 will be described in reference to the other figures.

A work station 15 for use by a professional, for example a radiologist, is connected to the computation platform 12. Hereafter, the terms “professional” and “user” will be used interchangeably. The work station 15 is configured to receive data and model calculations from the computation platform, and to send instructions and feedback to the computation platform 12. The work station 15 can visualize received raw data and model results.

In step S3, the professional selects the model (or in general: specifies model parameters) for use in a calculation. Based on the entered model parameters, in step S4 the platform 12 generates the model (if needed—the model may be already cached), performs the needed calculations for training the model (if needed—training data for the model may already be available in the computation platform 12), and applies the model to the imaging data that was received in step S2. In general, the computation platform will use stored results for calculations that have been performed earlier (i.e. calculated image features, model training data) and only perform the calculations it has not done before. This way, the professional accessing the computation platform 12 using the work station 15 can have a fast response to his or her instructions.

The result of the model calculations, for example classification of the most recent imaging data and associated patient metadata, is sent to the professional in step S5. The received data is visualized on the work station 15. The professional will examine the results and may prepare feedback in step S6. Feedback may for example be that, in the professional's opinion, the presented classification is correct or incorrect. In this manner, the feedback information can be used to enrich the model so that at a later stage more sophisticated models can be trained.

Along with the feedback, the source of the feedback may also be stored. That makes it possible to train future models using only feedback from selected sources. For example, the professional can request models that are only trained using his own data or data from close colleagues (e.g. “trusted data”). Instead or in addition to this, the feedback can be used to incrementally adjust the decisions functions of the model. The feedback can be used only in one or more selected decision functions, again to ensure that models are trained using data from known and trusted sources.

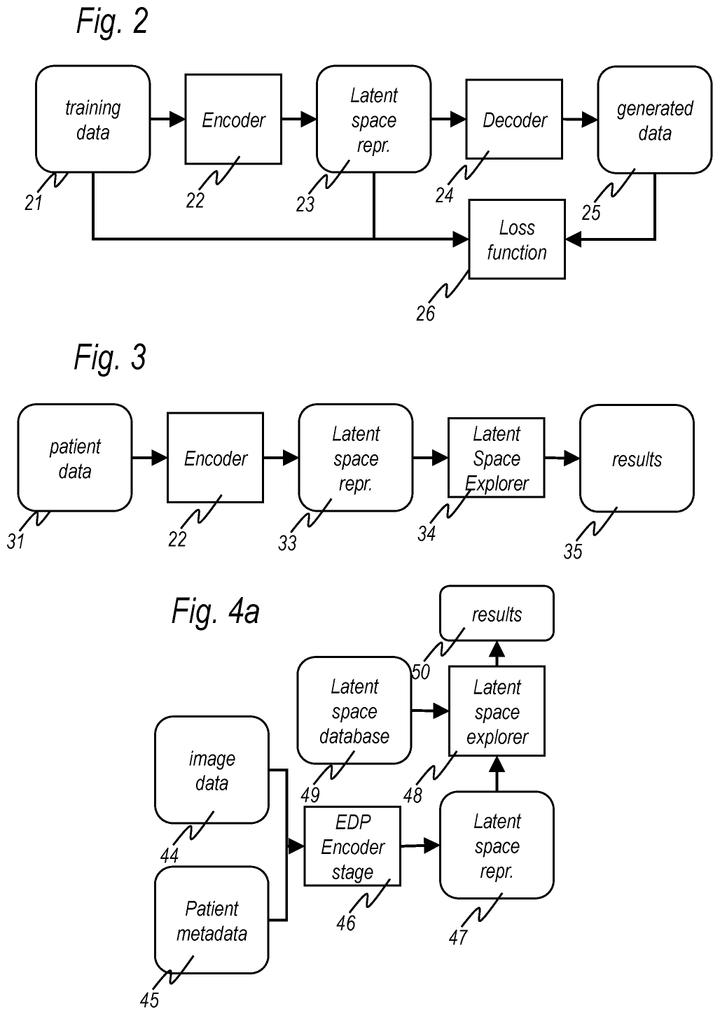

The model will now be further discussed in reference to FIGS. 2 and 3. FIG. 2 schematically shows a training stage of the model, and FIG. 3 schematically shows an application stage of the model.

The model can make use of an encoder-decoder pair (EDP). The encoder 22 is a neural network which takes data input x (e.g. training data 22 or patient data 31) and outputs a latent space or representation space value z (latent space representation 23, 33). The decoder 24, is also a neural network. It takes as input the latent space value z, and calculates an approximation of the input data x′ (generated data 25). The loss function 26 is designed to make the encoder and decoder work to minimize the difference between the actual and approximated inputs x and x′. A key aspect of the EDP is that the latent space z has a lower dimensionality than the input data. The latent space z is thus a bottleneck in the conversion of data x into x′, making it generally impossible to reproduce every detail of x exactly in x′. This bottleneck effectively forces the encoder/decoder pair to learn an ad-hoc compression algorithm that is suitable for the type of data x in the training set. Another way of looking at it, is that the encoder learns a mapping from the full space of x to a lower dimension manifold z that excludes the regions of the full space of x that contain (virtually) no data points.

An example EDP is an autoencoder. The most basic autoencoder has a loss function which, as a loss function 26, calculates an L1 or L2 norm of the generated data minus the training data. However, if the latent space is to have certain characteristics (such as smoothness), it is useful to also use aspects of the latent space as input in the loss function 26. For example, a variational autoencoder (Diederik P Kingma and Max Welling, “Auto-encoding variational Bayes”, Proceedings of the 2nd International Conference on Learning Representations, 2013) has a loss function that includes next to the standard reconstruction error an additional regularization term (the KL divergence) in order to encourage the encoder to provide a better organization of the latent space.

A feature of variational autoencoders is that, contrary to the most basic autoencoder, the latent space is stochastic. The latent variables are drawn from a prior p(z). The data x have a likelihood p(x | z) that is conditioned on the latent variables z. The encoder will learn a p(z | x) distribution.

In a further development of VAE's, a β parameter was introduced to add more weight to the KL divergence, in order to promote an even better organization of the latent space, at the cost of some increase in the reconstruction error.

Autoencoders and VAE's are not the only possible EDP's that can be used. It is also possible to use a U-Net as encoder-decoder. A U-Net EDP is similar to an EDP using a conventional Convolutional Neural Network encoder and decoder, with the difference that there are additional connections between encoder layers and the mirrored decoder layers, which bypass the latent space 23 between the encoder 22 and decoder 24. While it may seem counter-intuitive to have these latent space bypasses in order to promote a better latent space, these bypasses may actually help the encoder to reduce the reconstruction error without overburdening the latent space with storage of high-frequency image details which are important for the decoder to accurately recreate the input image (and thus to reduce the reconstruction error), but which are not important for the purposes of the latent space representation (more details on the purpose of the latent space representation are discussed in connection with the FIGS. 3-8).

As a further refinement, the encoder may be built using a probabilistic U-Net. A probabilistic U-Net is able to learn a distribution over possible outcomes (such as segmentation) rather than a single most likely outcome/segmentation. Like VAEs, the probabilistic U-Nets use a stochastic variable distribution to draw latent space samples from. The probabilistic U-Net allows for hi-resolution encoding/decoding without much loss in the decoded images. It also allows the variability in the labelled image or other data (due to radiologist marking variability, measurement variability, etc.) to be explicitly modelled.

Another way to improve the latent space representation 23 is by including a Discriminator of a Generative Adversarial Network (GAN) in the loss function 26. The discriminator is separately trained to learn to distinguish the generated data 25 from the original training data 21. The training process then involves training both the EDP and the loss function's discriminator. Usually, this is done by alternately training one and the other. Use of a GAN discriminator typically yields sharper and more realistic looking generated data than traditional reconstruction errors (e.g. L1 or L2 norm).

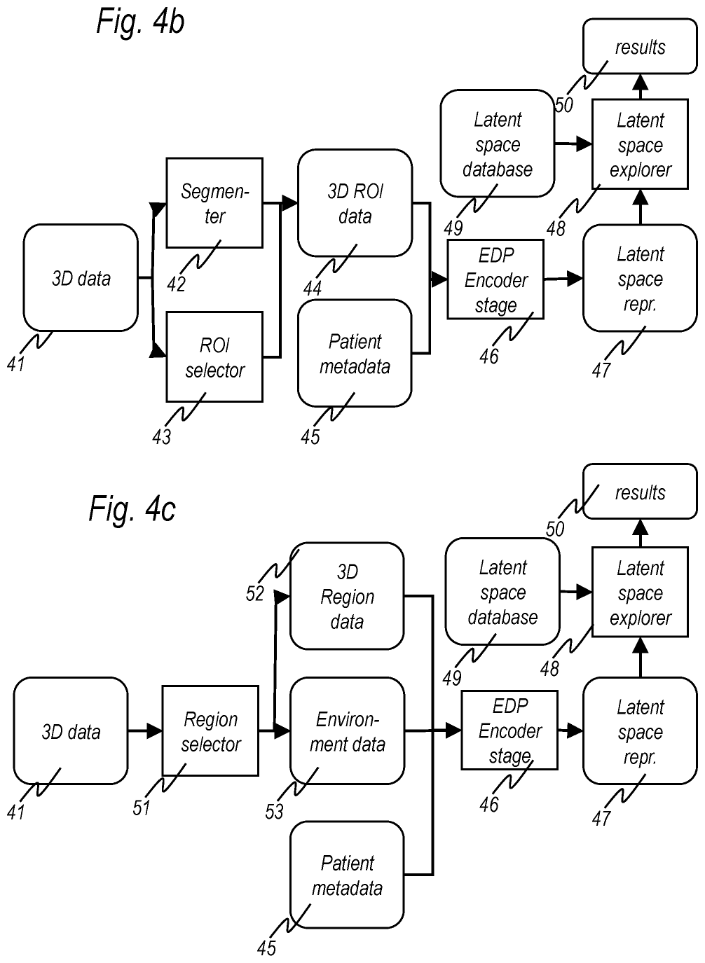

In FIG. 3, only the encoder 22 of the EDP is used. Instead of the decoder of FIG. 2, for new patient data a latent space explorer module 34 is used to determine resulting data 35 from the latent space representation 33. Further details of the latent space explorer module will be discussed in reference to FIGS. 4-7.

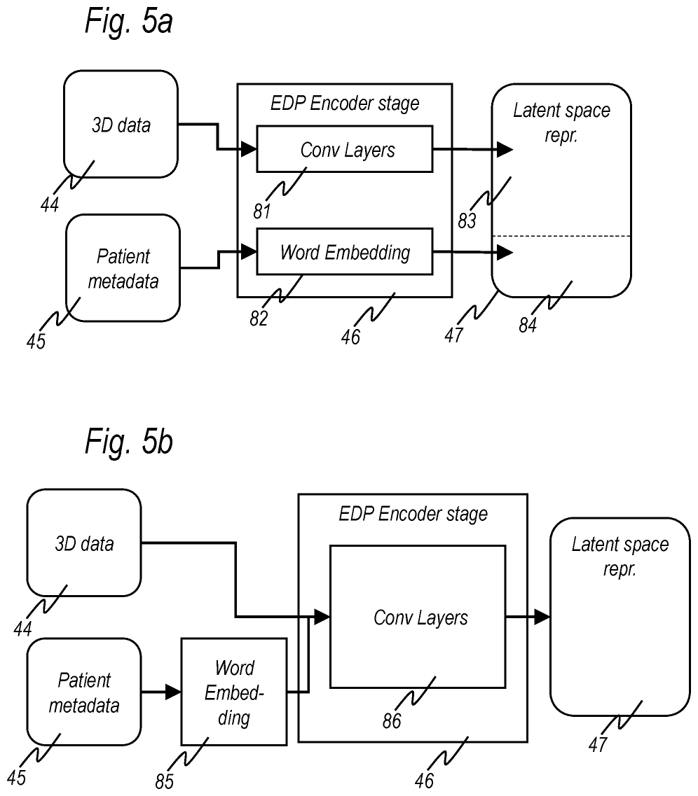

FIG. 4a shows a first example of a model according to an embodiment of the disclosed subject matter. The processing starts on the basis of imaging data 41, for example CT or MRI data. The data could have a resolution of 512×512×512 voxels or points. The imaging data 41 is provided to the encoder stage of the EDP, along with optional patient metadata 45. The patient metadata 45 may describe potentially relevant data such as age, gender, medical history items, health-related habits, etc. Such data is often in the form of numbers combined with text keywords. Using bag-of-words techniques or word embeddings, such data can be reduced to pure numbers for inclusion in the EDP encoder next to the 3D ROI data. More details are discussed in reference to FIG. 5a-b.

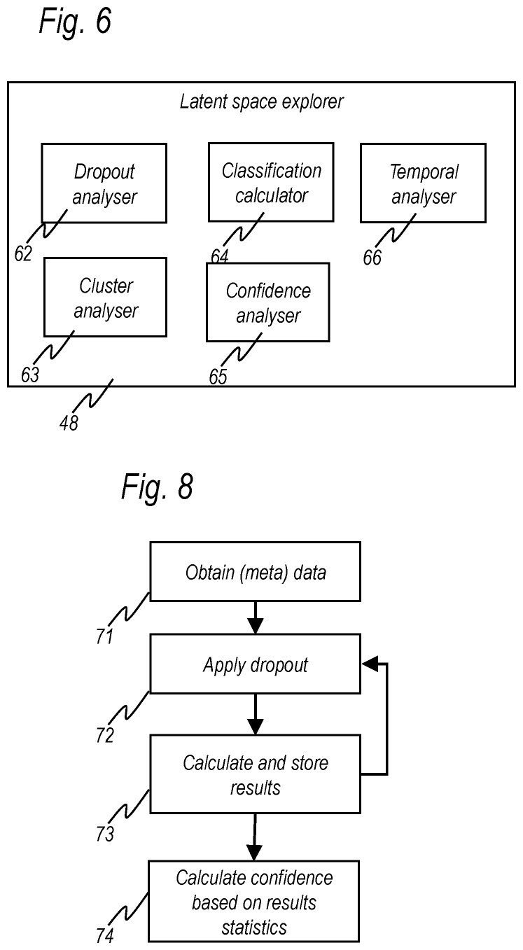

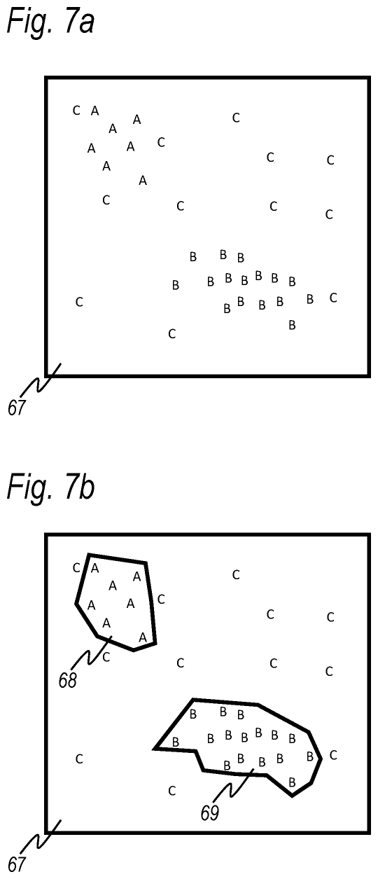

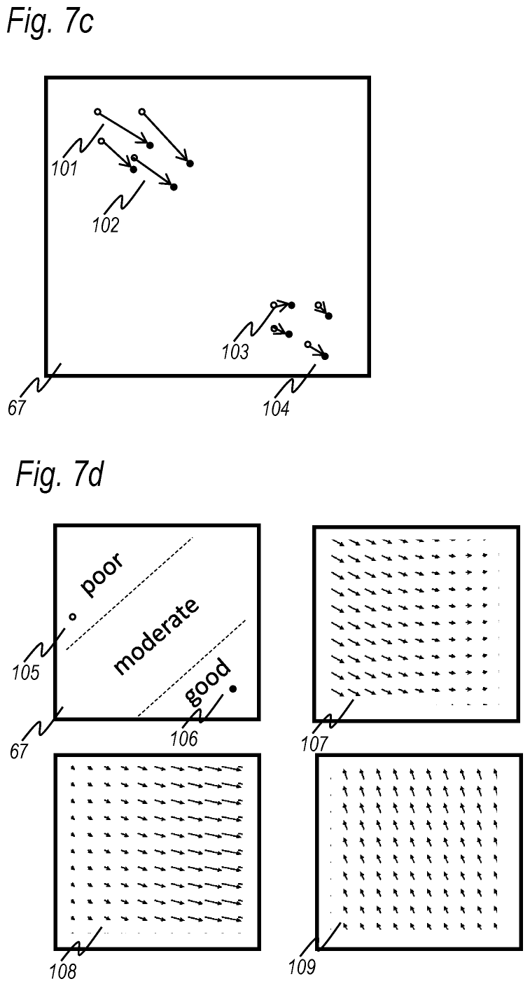

The EDP encoder stage 46 has already been discussed in reference to encoder 22 of FIG. 2. It typically uses a number of hidden layers to calculate the latent space representation 47. For example, the hidden layers may comprise convolutional layers, pooling layers, and fully connected layers. The latent space representation 47 is provided to a latent space explorer module 48, which will be further discussed in reference to FIGS. 6 and 7. Briefly put, the latent space explorer module 48 will analyze the latent space representation 47 of the current 3D and patient data 44, 45 in the context of earlier (classified) latent space representations of similar data, stored in the latent space database 49, in order to come up with a resulting classification 50. The latent space database 49 includes an encoding of patient data to the latent space, as well as patient metadata. It may also include temporal information, that is, the same patient's data as determined at different points in time. It may also include data relating to the patients treatment and eventual outcome.

FIG. 4b shows a variant of FIG. 4a. The processing again starts on the basis of 3D medical imaging data 41, for example CT or MRI data. The data could have a resolution of 512×512×512 voxels or points. While in principle the imaging data 41 could be directly provided to the encoder stage of the EDP, it is often useful to have the EDP process (both in training and in application) smaller volumes. To that end, a segmentation or segmenter module 42 may segment the 3D data (e.g. identify bone, various tissue types, organs, etc.). A region of interest (ROI) selector 43 may identify regions for closer examinations, such as regions that appear to contain a nodule or lesion. Data from one or both modules 42, 43 is used to determine 3D ROI data 44 to be used in the encoder of the EDP. For example, the 3D ROI data can be a block of 32×32×32 centered at a point where the segmenter 42 indicates that interesting tissue is to be found, and the ROI selector indicates a lesion might be present. The segmenter module 42 and ROI selector 43 preferably work in parallel, but a serial arrangement is possible as well.

FIG. 4c discloses a further variant of the model of FIG. 4b. Instead of using a segmenter 42 and an ROI selector 43, a region selector 51 is used which applies a type of multiscale approach. It will generate 3D region data 52 (e.g. the block of 32×32×32 points) and environment data 53, which is representative of the block's surroundings. The environment data 53 can include information concerning the general location of the block with respect to the full set of 3D data, distance to points of reference (e.g. distance to edge of the tissue), or other data useful for characterizing the location of the 3D region data 52 in the larger 3D data 41. The 3D region data 52, environment data 53 and patient metadata 45 is provided to the encoder stage of the EDP. The remainder of the processing is as described already in reference to FIGS. 4a and 4b.

FIGS. 5a and 5b show alternative ways in which the patient metadata can be included as input for the encoder stage 46. In the example of FIG. 5a, the EDP encoder stage comprises convolutional layers 81 for processing the 3D data 44 (which can also be the region data 52 and the environment data 53) and a word embedding module 82 for processing the patient metadata. The outputs 83 and 84 of the respective modules 81 and 82 are concatenated to form the latent space representation 47.

In FIG. 5b, the patient metadata is first converted to a reduced dimensionality using word embedding module 85. The output of the word embedding 85 is then fed into the EDP encoder stage 46 together with the mentioned 3D data to be processed by the convolutional layers 81 in order to obtain the latent space representation 47.

For patient metadata which is (close to) numeric in nature, such as age or gender, the word embedding module 85 in FIG. 5a can actually be a pass-through function. This type of metadata can be directly input to the encoder stage 46 which will encode it together with the convolutional layers to a latent encoding. Alternatively (FIG. 5b), if the patient data is more free text, as in a diagnosis text, etc., features need to be derived from this either before entering the EDP (as in word embedding 85 of FIG. 5b) or in the EDP encoder stage 46 (not shown in FIG. 5b). The concatenation of the metadata based features to the latent space representation may be done after dimensionality reduction/feature extraction of the latent space representation 47.

FIG. 6 schematically shows a latent space explorer module 48 according to an embodiment of the disclosed subject matter. The module may comprise a dropout analyzer 62, a cluster analyzer 63, a classification calculator 64, a confidence analyzer 65, and a temporal analyzer 66. All of these components are optional, though in combination they synergistically improve the working of the latent space explorer. The dropout analyzer 62 is described further in reference to FIG. 8.

The functioning of the various latent space explorer components is also discussed in reference to FIGS. 7a-7d. In said figures, the latent space is schematically represented as a 2D space, by rectangle 67. It should be noted that this is generally a simplification, useful to determine certain principles schematically. The latent space can be N dimensional, with N typically in the range 2-1000. However, in principle higher dimension latent spaces can be used. In addition, it should be noted that depending on the encoder type, the latent space may be stochastic in nature, for example having both an average (μ) and a standard deviation (σ).

Each latent space representation from the latent space database 49 is represented by a letter A, B, or C. The position of the letter represents the latent space representation (in this exemplary 2D example) and the letter itself represents a classification. For example, if the data relates to lesions, A, B and C might relate to different types of lesions.

The cluster analyzer 63 is configured to detect groups or clusters of points with like classification in the latent space 67. The encoder is trained in such a manner that images with related classifications are mapped in similar regions in latent space, so that there these similarly classified images can be found in groups or clusters according to a relevant distance metric of the latent space.

The cluster analyzer 63 may employ an algorithm such as k-means clustering. In the example of FIG. 7b, the cluster analyzer 63 has found a cluster 68 of A points and a cluster 69 of B points. The C points are not located in a single cluster, although they could be assigned to a number of separate clusters (not shown).

The 2D space is thus a projection of the latent space, with in it the classifications (or other labels of interest) of which the uncertainty may be calculated. That is, if part of the classification is a specific type of nodule, from the variance and overlap it can be seen if there is a need to sample more from this type of image data. The 2D (or N-dimensional) space can be any projection for any pathology classification that is of interest. These classifications can all be calculated via the same latent space manifold. Instead of a cluster analyzer 63, a different type of module can be used. What is key, is that the module identifies, for any given new data point, similar earlier data points with a known classification, so that a classification of the new data point may be arrived at.

Returning to FIG. 6, the classification calculator 64 is an optional component that evaluates the classifier function F(z) 55 if one has been trained as part of the training, to obtain a classification result. Such classification could be a prognosis as illustrated in FIG. 7d. In general, F(z) can be trained to generate any type of known parameter associated with patient data.

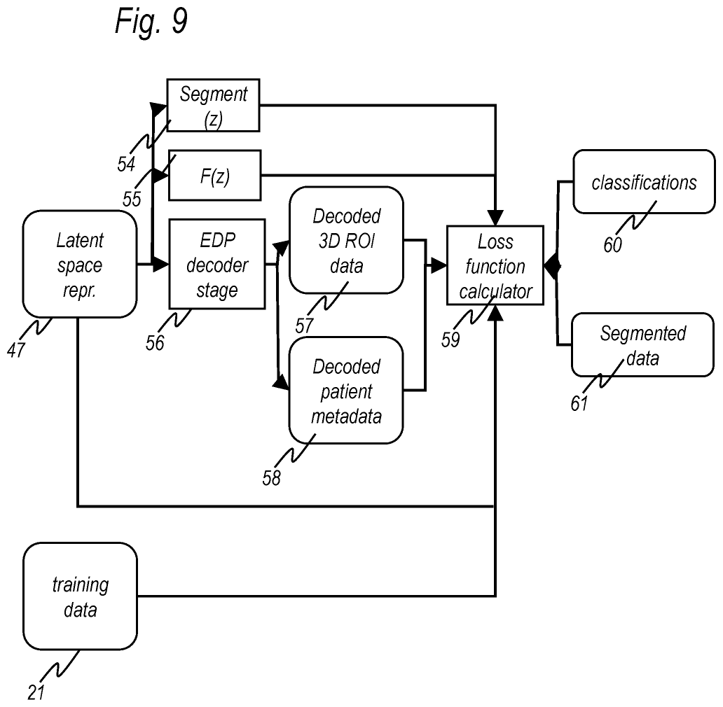

The confidence analyzer 65 may work as follows. First it determines if a current latent space representation 47 is located in a cluster of points with a specific classification. In case a classifier function F(z) 55 is available (see FIG. 9), it will check if the result of the classification calculator 64 matches the specific classification of the cluster. If the latent space representation 47 is in a cluster with specific classification and the result of the classification calculator 64 matches the specific classification of the cluster, then the confidence in the result is relatively high. If the latent space representation 47 is not in any cluster and not near earlier data points, then the result will be solely determined by the output of the classification calculator 64, and the confidence value will be moderate to low. If the latent space representation is in an area of the latent space close to many different classifications, the confidence will be low. If the latent space representation has a sigma value indicating a statistical property such as standard deviation of the stochastic latent space variables, the sigma value can also be taken into account by the confidence analyzer 65. A lower sigma value can indicate a higher confidence value.

The case where the latent space representation 47 is not in any cluster and thus not near earlier data points, is indicative of a situation where the latent space is not well-suited to distinguish the different classifications. It may be that the dimensionality of the latent space is too high or too low, or that important discriminating data is missing from the input. While that may be a result that is suboptimal from the point of view of promptly determining a classification, the fact that it is known that the classification is unreliable is worthwhile in itself. It could, for example, prompt the practitioner to schedule further tests in order to obtain a more reliable outcome. On the other hand, if the confidence analyzer declares a high confidence (because for example the point is in the middle of a homogenous cluster of points), it may tell the practitioner that further tests are not needed.

There are various ways to calculate a confidence value. Given the projection (interpreted as a probability distribution) and point x, if P(A | x) is close to 1, and P(B | x) and P(C | x) are both close to 0, that means that the assignment to A is quite confident. If P(A | x), P(B | x) and P(C | x) are all far from 0, then the assignment is less confident. Alternatively one could use a so-called silhouette-score which is effectively a distance to center divided by max spread, which will be much higher for A than for C.

The temporal analyzer 66 follows points in latent space over time. In FIG. 7c, an illustrative example is given. The numeral 101 refers to a first group of points (open circles) according to a first measurement. The numeral 102 refers to a second group of points (closed circles) according to a second measurement. There is a one-to-one correspondence between a point from the first group and a point from the second group That is, both points in each pair are based on data from the same patient, but measured at different moments in time. In the example of FIG. 7c, the data points 101 move to 102 over time, while the data points 103 move to 104 over time.

The temporal analyzer 66 is able to follow development over time in lateral space. This is useful, for example in order to determine the effect of a treatment. In the example of FIG. 7c, the latent space database 49 may have recorded that the patients of point group 101 have received treatment X, which caused a change in latent space to group 102. The patients of point group 103 may have received the same treatment X, resulting in a relatively smaller change to point group 104.

If enough of this statistical information is collected, it becomes possible to determine a vector F_{X}, including an uncertainty factor, which indicates the likely effect over time (in terms of movement in latent space) of treatment X. This can be extended to also include other treatments, say Y and Z.

This is illustrated in FIG. 7d. Initially, a patient's data (image data, patient metadata) results in a mapping to point 105 in latent space 67. That point is part of a latent space area or cluster with generally poor prognosis (this could be predicted by the F(z) function). A treatment which would cause the point 105 to move towards target point 106, in a part with generally good prognosis, would therefore likely improve this patient's health. The effects of treatment X for a number of points in latent space 67 are represented as vector field 107, the effects of treatment Y as vector field 108, and the effects of treatment Z (which in this case may represent the action of not doing anything) as vector field 109. Clearly, treatment Z is to be avoided, since it tends to move data points toward a region with poor prognosis, i.e. the condition generally worsens over time. Treatment X has a positive effect, which is more pronounced for points in the top left corner of the latent space and less pronounced in the middle and right hand side of the latent space. Treatment Y also has a positive effect, which is more pronounced in the middle and right hand side of the latent space. In the present case, the patient might benefit from first treatment X, until the data point has moved to the middle of the latent space, after which treatment Y would be more effective.

It should be noted that the vector fields of FIG. 7d are a simplification. The data is stochastic in nature, so any vector also has an uncertainty value. Still, the general principle holds that the temporal analyzer 66 can predict, based on previous measurements, which treatment is most likely to generate the most improvement, for any given momentary position in latent space.

It is also worth noting that the vector fields, and indeed the entire latent space and prognosis (or generally, classification) values, can be determined based on a subset of patent data, for example according to patient metadata. One can determine separate vector fields depending on factors such as age, sex, smoking habits, etc. These vector fields can be seen as a summary of all or a subset of the data that is available in the latent space database 49.

FIG. 8 schematically shows a method of performing dropout analysis. The dropout analyzer 62 of FIG. 5 could be configured to perform this method. The method involves analyzing changes in the latent space representation 47 in response to small changes (e.g. deletions) in the input data, which is obtained in step 71. The small changes performed in the “apply dropout” step 72 could be setting the value of one or more 3D input pixels to a predetermined value or setting one or more patient metadata settings to a predetermined value. In step 73, the encoder stage is used to calculate the latent space representation for the modified input. For example, by changing the age of the patient, it is possible to analyze how sensitive the latent space representation is to the factor age. The dropout analyzer 62 can thus be used to determine, in step 74, a confidence value of the latent space representation 47 and, by extension, of the result 50 that is determined from the latent space representation.

The influence of the randomness of dropout on the resulting classification, combined with the robustness of the features will lead to an observed confidence. It might be that this confidence boundary might be very sharp, which then also shows that the predictions are stable.

FIG. 9 discloses a decoder stage of the EDP and a loss function to be used in training the model. It can be used together with an encoder such as those shown in FIG. 4a, 4b or 4c, and starts from the shared latent space representation 47. The EDP decoder stage 56 will be trained to generate decoded 3D data 57 which resembles either the 3D ROI data 44 of FIG. 4a or the 3D region data 52 and environment data 53 of FIG. 4b. In addition, it will generate the patient metadata 58 to be compared with the original patient metadata 45.

The loss function calculator 59, which steers the training of the encoder 46 and decoder 56, will be provided with the original data 21, or 44 and 45 or 52, 53, and 45 and the generated data 57, 58. In addition, it may optionally be provided with the output of classifier function F(z) 55 and the known classification 60. By including the classification prediction in the loss function, the encoder may be encouraged to better distinguish classifications in the latent space.

Optionally, the loss function can also be provided with the output of segmentation function 54 and known segmentation data 61. In this way, a classifier function 55 and segmenter function 54 may also be trained during the training phase. The loss function may also receive the latent space representation as input, for example in order to calculate a latent space regularization term.

The loss function calculator can be a determined loss function, such as L2 or L1 for optimizing the EDP. Alternatively, this loss function could be based on the underlying distribution and use a GAN to learn a loss function.

The classifier function F(z) 55 may be a deep neural network, such as a CNN. The segmenter function 54 may also be a deep neural network, such as a CNN.

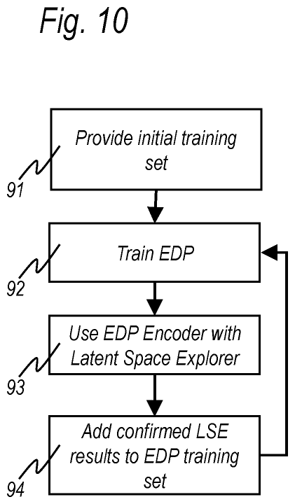

FIG. 10 schematically shows an iterative method according to an embodiment of the disclosed subject matter. In step 91, an initial training set is provided, which is used to train the EDP in step 92. In step 93, the EDP is used together with the latent space explorer module 48 to analyze new imaging and patient data. In step 94, the results obtained in the analysis are approved (or corrected by a practitioner and then approved) for addition to the set of training data. Control passes back to step 92, where the EDP is incrementally trained or completely retrained, taking the new training data into account.

Combinations of specific features of various aspects of the disclosure may be made. An aspect of the disclosure may be further advantageously enhanced by adding a feature that was described in relation to another aspect of the disclosure.

It is to be understood that the disclosure is limited by the annexed claims and its technical equivalents only. In this document and in its claims, the verb “to comprise” and its conjugations are used in their non-limiting sense to mean that items following the word are included, without excluding items not specifically mentioned. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

We claim:

1. A computer-implemented method for processing medical image data, the method comprising:
inputting, with one or more processors of one or more computation devices, medical image data into an encoder stage of an encoder-decoder pair (EDP) as a first input among one or more inputs;
calculating, with the one or more processors, a latent space representation of the one or more inputs using the encoder stage of the EDP;
providing, from a latent space database stored within one or more storage devices accessible by the one or more computation devices, latent space representations of other inputs; and
determining, with the one or more processors, a classification based on the latent space representation of the one or more inputs and at least one latent space representation of the other inputs.

2. The method of claim 1, further comprising inputting, with the one or more processors, patient metadata into the encoder stage of the EDP as second input.

3. The method of claim 2, wherein the patient metadata and the calculated latent space representation are added to the latent space database stored within the one or more storage devices.

4. The method of claim 1, further comprising projecting, with the one or more processors, the latent space representation using a projection function to obtain a classification value.

5. The method of claim 4, wherein the projection function uses a convolutional neural network (CNN).

6. The method of claim 1, further comprising determining, with the one or more processors, if the latent space representation is part of a cluster of other latent space representations using a cluster detection algorithm.

7. The method of claim 1, wherein the EDP is a variational autoencoder.

8. The method of claim 7, further comprising training, with the one or more processors, the EDP using a loss function based on a Generative Adversarial Network (GAN).

9. The method of claim 1, wherein a plurality of medical image data over time are used as input, and further comprising tracking, with the one or more processors, the corresponding latent space representations over time.

10. A computer-implemented method of training a model using medical image data, the method comprising:
inputting, with one or more processors of one or more computation devices, medical image data into an encoder-decoder pair (EDP) as first input among one or more inputs;
training, with the one or more processors, the EDP to reproduce the one or more inputs, whereby between an encoder part of the EDP and a decoder part of the EDP, the encoded data is represented in a latent space; and
further training, with the one or more processors, a projection function for projecting the latent space representation to a classification value,
wherein a loss function of the EDP training is based on the projected classification.

11. The method of claim 10, wherein patient metadata is input into the EDP as second input.

12. The method of claim 11, wherein the patient metadata is included as an embedding layer.

13. The method of claim 10, wherein the projection function uses a convolutional neural network (CNN).

14. A computing system for processing medical image data, comprising:
one or more computation devices in a computing environment and one or more storage devices accessible by the one or more computation devices, wherein the one or more computing devices comprise one or more processors, and wherein the one or more processors are programmed to:
input medical image data into an encoder stage of an encoder-decoder pair (EDP) as a first input among one or more inputs;
calculate a latent space representation based on the one or more inputs using the encoder stage of the EDP;
provide, from a latent space database stored within the one or more storage devices, latent space representations of other inputs; and
determine a classification based on the latent space representation of the one or more inputs and at least one latent space representation of the other inputs.

15. The computer system of claim 14, and wherein the one or more processors are further programmed to input patient metadata into the encoder stage of the EDP as second input among the one or more inputs.

16. The system of claim 14, and wherein the one or more processors are further programmed to use a cluster detection algorithm to determine if the latent space representation is part of a cluster of other latent space representations.

17. The system of claim 14, wherein the EDP is a variational autoencoder.

18. The system of claim 14, wherein the EDP is trained using a loss function based on a Generative Adversarial Network (GAN).

19. The system of claim 14, wherein the EDP comprises a probabilistic U-Net.

20. The system of claim 14, and wherein the one or more processors are further programmed to track a latent space representation based on the one or more inputs over time.

21. A non-transitory computer-readable medium with instructions stored thereon, that when executed by one or more processors, perform the steps comprising:
inputting medical image data into an encoder stage of an encoder-decoder pair (EDP) as a first input among one or more inputs;
calculating a latent space representation of the one or more inputs using the encoder stage of the EDP;
providing from a latent space database latent space representations of other inputs; and
determining a classification based on the latent space representation of the one or more inputs and at least one latent space representation of the other inputs.
